(19) United States
(12) Patent Application Publication
(10) Pub. No.: US 2023/0181266 A1
(43) Pub. Date: Jun. 15, 2023

(54) AUTOMATIC SURGICAL SYSTEM SETUP AND CONFIGURATION

(71) Applicant: Alcon Inc., Fribourg (CH)

(72) Inventors: Paul R. Hallen, Colleyville, TX (US); Mark Alan Hopkins, Mission Viejo, CA (US)

(21) Appl. No.: 18/062,620

(22) Filed: Dec. 7, 2022

Related U.S. Application Data
(60) Provisional application No. 63/265,168, filed on Dec. 9, 2021.

(51) Int. Cl. A61B 34/00; G06F 21/35; G06F 21/32; G06V 40/16; G06V 20/64; G06K 19/07

(52) U.S. Cl. CPC A61B 34/25; G06F 21/35; G06F 21/32; G06V 40/172; G06V 20/64; G06K 19/0723; A61B 2034/258

(57) ABSTRACT

Systems are provided for automatically configuring devices and systems in an operating environment, e.g., an ophthalmic operating environment, to a user's desired settings. The system includes a controller configured to identify a user, e.g., a surgeon, via the utilization of a user-identifying identifier, e.g., a radio-frequency identification (RFID) device, which may communicate with a receiver operably coupled to the controller. The controller may further be configured to identify a surgical device, e.g., an ophthalmic probe, being used by the user during a surgical procedure via a device-associated identifier, e.g., a device-specific RFID device. Upon identification of the user and the surgical device, the controller may map the user and surgical device to one or more sets of predefined and user-input parameters/settings, and further configure the surgical device based on the mapped one or more sets of previously-determined parameters/settings.

### PRIORITY CLAIM

This application claims the benefit of priority of U.S. Provisional Patent Application Ser. No. 63/265,168 titled “AUTOMATIC SURGICAL SYSTEM SETUP AND CONFIGURATION,” filed on Dec. 9, 2021, whose inventors are Paul R. Hallen and Mark Alan Hopkins, which is hereby incorporated by reference in its entirety as though fully and completely set forth herein.

### BACKGROUND

The number and complexity of surgical procedures, including ophthalmic procedures such as vitreoretinal surgery, is increasing every day. Many such surgical procedures require the use of multiple devices, such as a microscope, display device, and various surgical tools and/or systems (e.g., consoles). For example, when performing a vitrectomy surgery, a surgeon may generally use a vitrectomy probe (e.g., a vitreous cutter), an infusion cannula, and an endoilluminator, which may all be in communication with a surgical console, in addition to other tools and devices.

Prior to or during performance of a surgical procedure, the surgical console may require certain inputs from the surgeon or other operating staff to drive one or more appropriate surgical tools for the procedure. For example, operation mode settings, user-preferred tool settings and parameters (e.g., power, duration, etc.), display settings, and other such inputs may need to be entered and/or confirmed prior to or during utilization of a surgical tool. Typically, a user, e.g., a surgeon, manually enters these various inputs, e.g., via a graphical user interface (GUI) on the surgical console or other interfaces. As a result, the flow of a surgical procedure may thus be disrupted, and the efficiency and time-management of the surgeon may be reduced.

Accordingly, there is a need in the art for improved devices, systems, and methods for streamlining the configuration of a surgical console to a user's desired settings during or in preparation for a surgical procedure.

### SUMMARY

The present disclosure relates to surgical devices, systems, and methods, and more particularly, to devices, systems, and methods for automatic setup and mode switching of surgical consoles and systems.

According to certain embodiments, a system for configuring a surgical console in a surgical operating environment is provided. The system includes a memory comprising executable instructions, and a processor in data communication with the memory. The processor is further configured to execute the instructions to cause the system to receive user-identifying information associated with a user in the surgical operating environment, map the user-identifying information to a user profile, identify, based on the user profile, one or more parameters for driving a surgical tool, and configure the surgical console to drive the surgical tool based on the one or more parameters.

### BRIEF DESCRIPTION OF THE DRAWINGS

So that the manner in which the above-recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 illustrates an operating environment with various surgical devices and systems whose settings may be auto-configured, in accordance with certain embodiments of the present disclosure.

FIG. 2A illustrates a flow diagram of a method for using a surgical configuration system, in accordance with certain embodiments of the present disclosure.

FIG. 2B illustrates a schematic diagram of a surgical configuration system, in accordance with certain embodiments of the present disclosure.

FIG. 3 illustrates exemplary components of the surgical configuration system of FIG. 2, in accordance with certain embodiments of the present disclosure.

To facilitate understanding, identical reference numerals have been used, where possible, to designate identical elements that are common to the Figures. It is contemplated that elements and features of one embodiment may be beneficially incorporated in other embodiments without further recitation.

### DETAILED DESCRIPTION

In the following description, details are set forth by way of example to facilitate an understanding of the disclosed subject matter. It should be apparent to a person of ordinary skill in the field, however, that the disclosed implementations are exemplary and not exhaustive of all possible implementations. Thus, it should be understood that reference to the described examples is not intended to limit the scope of the disclosure. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one implementation may be combined with the features, components, and/or steps described with respect to other implementations of the present disclosure.

Embodiments of the present disclosure generally relate to systems for automatically configuring surgical systems, such as surgical consoles, in an operating environment, e.g., an ophthalmic operating environment, to a user's desired settings. In certain aspects, the system includes a controller configured to identify a user, such as a surgeon, via the utilization of a user-specific portable component, e.g., a radio-frequency identification (RFID) device, which may communicate with a receiver operably coupled to the controller. Upon identification of the user, the controller may map the user to one or more sets of defined parameters/settings for the surgical system. The controller may further be configured to identify a surgical tool, such as an ophthalmic probe, to be used or being used by the user during a surgical procedure via, e.g., a tool-specific RFID device or other sensor. In certain aspects, upon identification of the surgical tool being used, the controller may place the surgical console in an appropriate operation mode associated with the surgical tool. In certain aspects, upon identification of the user and the surgical tool, the controller may cause the surgical console to drive the surgical tool based on the one or more sets of defined parameters/settings associated mapped with the user. In further aspects, the controller may be configured to identify the user and/or the surgical tool via image-recognition mechanisms.

As used herein, the term “surgical system” may refer to any surgical system, console, or device for performing a surgical procedure. For example, the term “surgical system” may refer to a surgical console, such as a phacoemulsification console, a vitrectomy console, a laser system, or any other consoles, systems, or devices used in an ophthalmic operating room, as known to one of ordinary skill in the art. Note that although certain embodiments herein are described in relation to ophthalmic systems, tools, and environments, the embodiments described herein are similarly applicable to other types of medical or surgical systems, tools, and environments.

As used herein, the term “sensor” may refer to any type of device that detects or measures, e.g., a physical input, and records, indicates, or otherwise responds to the physical input. For example, the term “sensor” may refer to a device configured to detect or measure a position, location, proximity (e.g., to a surgical console), tilt, height, speed (e.g., an accelerometer), temperature, etc., of a surgical tool, system, or user. In certain examples, the term “sensor” may refer to a device configured to detect touch, i.e., touching of a surgical tool or system by a user, such as a capacitive- or resistive-type touch sensor. In certain examples, the term “sensor” may refer to an imaging device configured to detect and relay image-based information, such as a charge-coupled device (CCD) or an active-pixel sensor (APS), such as a complementary metal-oxide-semiconductor (CMOS) sensor.

Although generally described with reference to ophthalmic surgical devices and systems, the devices and systems described herein may be implemented with other devices and systems, such as devices and systems for other surgeries, without departing from the scope of the present application.

As used herein, the term “about” may refer to a +/−10% variation from the nominal value. It is to be understood that such a variation can be included in any value provided herein.

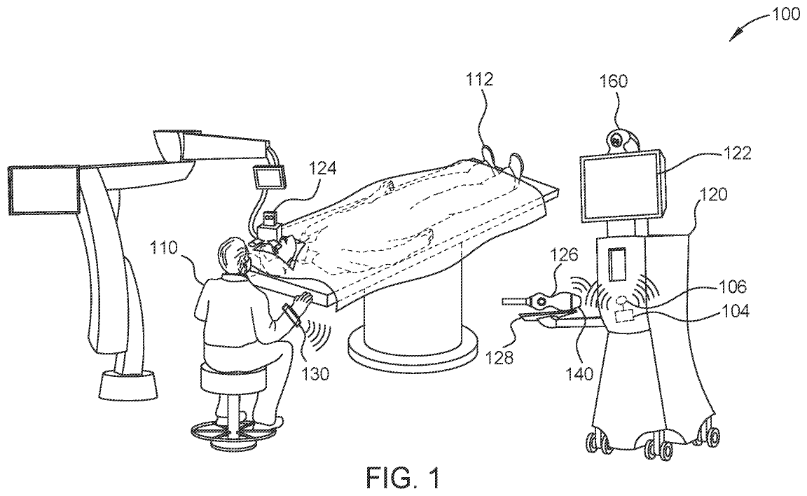

FIG. 1 illustrates an example of operating environment 100, such as an ophthalmic operating environment, in which surgical console 120 may be utilized for performance of a surgical procedure, according to embodiments of the present disclosure. As shown, operating environment 100 further includes surgeon 110, patient 112, as well as a plurality of surgical systems and tools, such as surgical console 120 having a display device 122, microscope system 124, and surgical tool 126. Generally, examples of suitable surgical systems that may be included in operating environment 100 include surgical devices and consoles for performing vitreoretinal procedures, cataract surgeries, corneal transplants, glaucoma surgeries, LASIK surgeries, refractive lens exchanges, trabeculectomies, keratotomy procedures, and keratoplasty surgeries, or other devices and consoles identifiable by those of ordinary skill. Consoles that are capable of performing two or more of these procedures are also within the scope of this disclosure. An example of a console configured for performing vitreoretinal procedures is the Constellation® System available from Alcon Laboratories, Inc., Fort Worth, Tex. An example of a console configured for performing cataract surgeries is the Centurion® System available from Alcon Laboratories, Inc., Fort Worth, Tex.

Surgical console 120 includes controller 104 (shown in phantom), and in certain embodiments, receiver 106 in communication with controller 104. Controller 104 is configured to cause surgical console 120 to perform one or more tasks for driving a surgical tool, e.g., surgical tool 126, according to stored settings and parameters associated with the surgical tool. Receiver 106 may include any suitable interface for communication (e.g., one-way or two-way signals) between controller 104 and, e.g., user identifier 130 discussed below. For example, receiver 106 may include a wireless or wired connection between controller 104 and user identifier 130. In certain embodiments, receiver 106 is in further communication (e.g., one-way or two-way signals) between controller 104 and tool identifier 140, and/or a usage sensor, each described in further detail below.

In certain embodiments, receiver 106 includes an RFID reader, a Bluetooth receiver, a near field communication (NFC) reader, or another similar wireless-type receiver. In the embodiments of FIG. 1, controller 104 and receiver 106 are integrated within surgical console 120, wherein controller 104 includes or refers to one or more processors and/or memory devices integrated within the surgical console. In certain other embodiments, controller 104 and/or receiver 106 are stand-alone devices or modules that are in wireless or wired communication with, e.g., surgical console 120 and other devices within operating environment 100. In certain embodiments, controller 104 refers to a set of software instructions that a processor associated with surgical console 120 is configured to execute. In certain aspects, operations of controller 104 may be executed partly by the processor associated with controller 104 and/or surgical console 120 and partly in a public or private cloud.

In the example of FIG. 1, user identifier 130 is depicted on surgeon 110. User identifier 130 generally includes any suitable article, device, or component that may signal to controller 104 the presence of surgeon 110 in operating environment 100 and further identify surgeon 110 for purposes of automatic setup and configuration of surgical console 120 according to defined parameters/settings associated with surgeon 110. In certain embodiments, user identifier 130 includes a user-specific digital or analog barcode or other form of machine-readable code (e.g., a Quick Response “QR” code) that may be captured or read by, e.g., an image scanner on surgical console 120 that is in communication with controller 104, which then utilizes the code to identify surgeon 110. In certain embodiments, user identifier 130 includes a device configured to wirelessly transmit user-specific identity data to, e.g., receiver 106 in communication with controller 104, for identification thereby. For example, user identifier 130 may include a passive or active RFID transponder or a similar device for transmitting (i.e., communicating) user-specific identity data to receiver 106, which may include an RFID receiver. Specific examples of user identifier 130 may include an employee badge having an analog machine-readable code, an employee badge or tag having an RFID transponder, a bracelet having an RFID transponder, etc.

In certain embodiments, user identifier 130 may be a device configured with wireless communications capabilities, such as hardware/software for communicating user-specific identity data to controller 104. For example, user identifier 130 may include a wireless cellular device, such as a smart phone, or a similar smart device, such as a smart watch, tablet, or any other electronic device capable of transmitting user-specific identity data to controller 104 using technologies such as near field communications, Bluetooth, or WiFi. In certain embodiments, the smart device may execute a software application that causes the smart device to communicate user-specific identity data to controller 104 when surgeon 110 is in proximity to surgical console 120. For example, when the surgeon 110 is in proximity to surgical console 120, the software application may be configured to communicate with controller 104 either automatically or as a result of some user action (e.g., user input). In certain embodiments, user identifier 130 may require surgeon 110 to enter a user-specific password to activate or unlock user identifier 130 and enable communication between user identifier 130 and, e.g., controller 104 or surgical console 120. In certain embodiments, in addition to identity data, user identifier 130 may also provide user-associated and user-preferred surgical tool and/or system settings, operation modes, operation and/or tool sub-modes, task parameters, calibration data, and the like, to controller 104.

During performance of an ophthalmic surgical procedure on patient 112, surgeon 110 may utilize one or more surgical tools, including surgical tool 126. As described above, surgical tool 126 may include any suitable tool for performing an ophthalmic procedure, e.g., vitreoretinal surgery, cataract surgery, glaucoma surgery, etc. In certain embodiments, surgical tool 126 and/or surgical console 120 may include a tool identifier 140. Tool identifier 140 includes any suitable device or component that may identify or indicate the type of surgical tool 126 to controller 104 for purposes of automatic setup, configuration, and/or operation mode selection of surgical console 120 according to defined parameters/settings associated with surgical tool 126. In certain embodiments, tool identifier 140 further includes a usage sensor, which may be in direct or indirect communication with controller 104. The usage sensor may include any suitable type of sensor for detecting usage or handling of surgical tool 126 by surgeon 110. For example, in certain embodiments, the usage sensor includes a touch or pressure sensor on surgical tool 126 configured to detect handling of surgical tool 126 by surgeon 110, such as a capacitive, inductive, resistive, or piezoelectric sensor disposed on a handle of surgical tool 126. In certain embodiments, the usage sensor includes, e.g., an accelerometer or tilt sensor on surgical tool 126 configured to detect movement of surgical tool 126. In further embodiments, the usage sensor includes a proximity or location sensor on surgical tool 126 configured to detect a locus of surgical tool 126, e.g., relative to user identifier 130, surgical console 120, and/or operating environment 100.

In embodiments where surgical tool 126 is in wired communication with surgical console 120 and/or controller 104, tool identifier 140 may transmit a tool-specific signal, through wired connection, to controller 104 to identify surgical tool 126, upon detection of usage or handling of surgical tool 126, e.g., by the usage sensor. In certain embodiments, tool identifier 140 may transmit a tool-specific signal, through wired connection, to controller 104 upon activation of tool identifier 140 or surgical tool 126. In such embodiments, the tool-specific signal may indicate both the identity (e.g., type of tool, model, etc.) of surgical tool 126 and usage or handling thereof, and may or may not be based on detection of handling by a usage sensor.

Similarly, in embodiments where surgical tool 126 is in wireless communication with surgical console 120 and/or controller 104 via receiver 106, tool identifier 140 may transmit a tool-specific signal, through wireless connection, to controller 104 via receiver 106 to identify surgical tool 126, upon detection of usage or handling of surgical tool 126, or upon activation of tool identifier 140 or surgical tool 126 (which may or may not be based on detection of handling by a usage sensor). In certain embodiments, surgical tool 126 may include (e.g., as part of tool identifier 140 or separately) an RFID transponder or similar component for wirelessly transmitting tool-specific identity data to controller 104 via receiver 106. In certain embodiments, tool identifier 140 includes a tool-specific barcode or other form of machine-readable code that may be captured or read by, e.g., an image scanner or similar device coupled to surgical console 120 and in communication with controller 104, which then utilizes the code to identify surgical tool 126. In such embodiments, usage of surgical tool 126 may be assumed by scanning of the code by, e.g., surgeon 110.

In certain embodiments, the usage sensor includes a laser sensor or similar device that is positioned on surgical console 120 and is configured to detect removal of surgical tool 126 from surgical console 120. In further embodiments, the usage sensor includes a weight sensor or other type of load cell disposed on surgical console 120, such as on tool tray 128 of surgical console 120, configured to detect lifting of surgical tool 126 from surgical console 120. In embodiments where the usage sensor is a laser sensor or weight sensor on surgical console 120, the usage sensor may detect usage of surgical tool 126 and send a tool-specific signal to controller 104, thereby indicating to controller 104 both the identity of surgical tool 126 and handling thereof by surgeon 110.

In certain embodiments, operating environment 100 further includes imaging device 160, which may be in direct or indirect communication with controller 104. Imaging device 160 may be any suitable type of imaging device configured to capture and relay images of, e.g., surgeon 110 and/or surgical tool 126, to controller 104 for purposes of identification of surgeon 110 and/or surgical tool 126 via image recognition processes. Thus, image recognition via imaging device 160 and controller 104 may be utilized to identify the presence and determine the identity of surgeon 110 in operating environment 100, as well as identify the type of tool and usage (e.g., by surgeon 110) of surgical tool 126, in place of or in addition to user identifier 130 and/or tool identifier 140. In certain embodiments, imaging device 160 includes a digital camera utilizing a charge-coupled device (CCD) or complementary metal-oxide semiconductor (CMOS) imaging sensor. Generally, imaging device 160 may be physically coupled to any suitable instrument or device within operating environment 100. In the embodiment of FIG. 1, imaging device 160 is coupled to surgical console 120, and may image both surgeon 110 and surgical tool 126 when picked up by surgeon 110. In further embodiments, imaging device 160 is coupled to, e.g., a wrist of surgeon 110, and is thus configured to capture images of and detect when surgeon 110 picks up surgical tool 126. In certain embodiments, multiple imaging devices 160 may be used in combination. For example, a first imaging device 160 on surgical console 120 may detect and capture images of surgeon 110 for image recognition of surgeon 110, and a second imaging device 160 on the surgeon's wrist may detect and capture images of surgical tool 126 for image recognition of surgical tool 126. In certain embodiments, imaging device(s) 160 facilitate a two-factor parity check for both presence of surgeon 110 within operation environment 100, as well whether surgeon 110 is using and/or handling surgical tool 126.

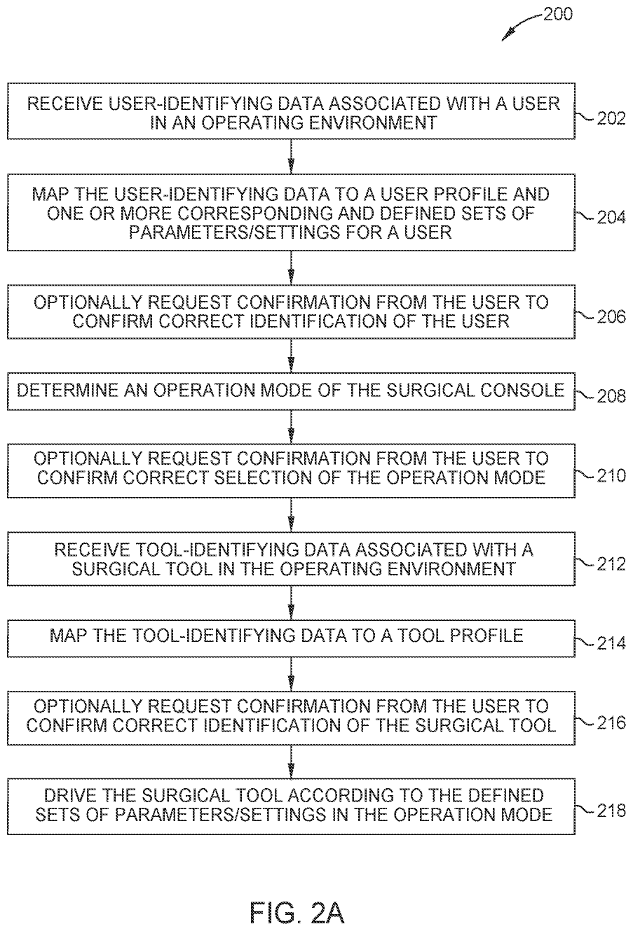

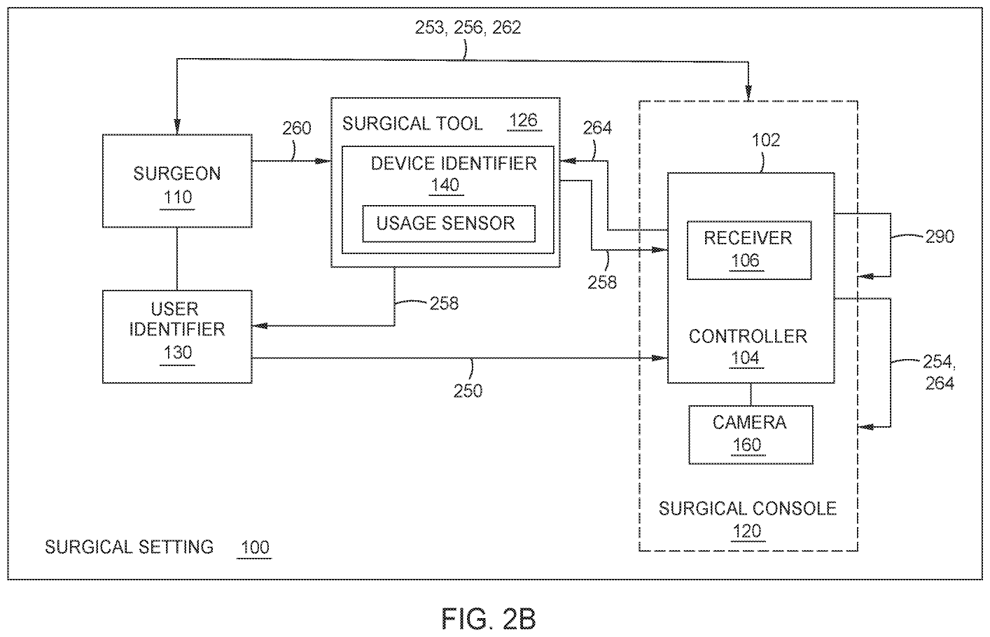

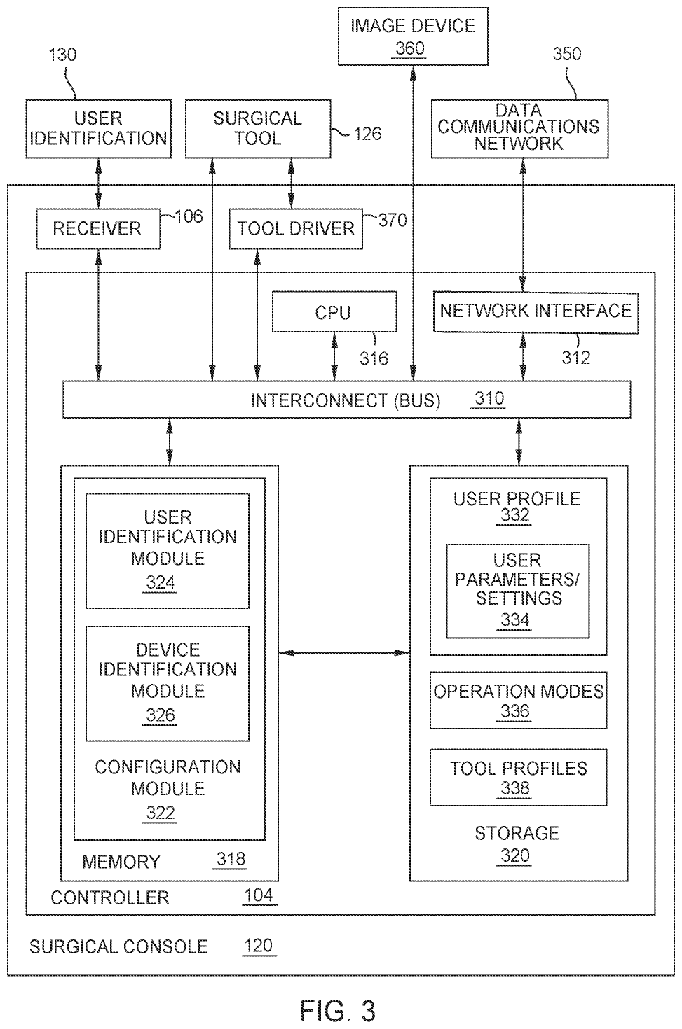

As discussed in greater detail below with reference to FIG. 2 and FIG. 3, controller 104 interfaces (e.g., wirelessly or wired) with, e.g., user identifier 130, tool identifier 140, and/or imaging device 160, to determine the identity of surgeon 110 and identify a tool used by surgeon 110 during an ophthalmic procedure. Upon determining the identity of surgeon 110 and/or surgical tool 126, controller 104 takes one or more actions for automatically configuring surgical console 120 according to defined (e.g., preset or predetermined) and user-associated surgical tool and/or system settings, operation modes, operation and/or tool sub-modes, task parameters, calibration data, and the like. Accordingly, controller 104 enables essentially “hands-free” system setup and configuration, thereby improving surgical procedure efficiency by reducing the amount of manual configuring required of surgeon 110 or other operating staff, who may have their hands already occupied with other tools or tasks.

FIG. 2A illustrates a flow diagram of a method 200 for automatically configuring a surgical console, e.g., surgical console 120, based on defined and user-associated tool and/or system settings, using various devices of FIG. 1, according to certain embodiments of the present disclosure. FIG. 2B illustrates a schematic diagram of various components of FIG. 1 used during method 200, according to certain embodiments of the present disclosure. Accordingly, FIGS. 2A-2B are described with reference to various components of FIG. 1 for clarity. Note that although the proceeding operations are described with reference to a single surgical tool 126, multiple surgical tools 126 may be utilized in combination with the systems and methods described herein. Further, when multiple surgical tools are utilized with method 200, the surgical tools may be different types of tools.

At operation 202, a controller associated with a surgical console receives user-identifying data associated with a user in a surgical operating environment. For example, controller 104 of surgical console 120 receives surgeon-identifying data about a surgeon 110 in the operating environment 100. As described above, in certain embodiments, controller 104 receives user-identifying data about the surgeon 110 from user identifier 130 associated with surgeon 110. In one example, when user identifier 130 is present in operating environment 100 and proximate to surgical console 120, user identifier 130 transmits user-identifying signal 250 (e.g., an RF signal) to controller 104. User-identifying signal 250 transmitted to controller 104 indicates to controller 104 the presence of surgeon 110 in operating environment 100 and identifies surgeon 110. In certain examples, user identifier 130 may transmit user-identifying signal 250 (e.g., WiFi signal) to controller 104 with or without surgeon 110 and user identifier 130 being or having to be in operating environment 100. In such examples, user-identifying signal 250 transmitted to controller 104 indicates to controller 104 the current or future presence of surgeon 110 in operating environment 100 and indicates the identity of surgeon 110.

As described above, user identifier 130 includes any suitable article, device, or component that may provide user-identifying signal 250 to controller 104 relating to the identity of surgeon 110 for purposes of setup and configuration of surgical console 120, according to defined parameters/settings associated with surgeon 110 for surgical tool 126. In certain examples, user identifier 130 is a passive or active RFID-type transponder that interfaces with controller 104 via receiver 106, which may be an RFID-type receiver, such as an RFID-type receiver configured to activate a passive RFID-type user identifier 130. In such embodiments, user identifier 130 may be brought into close proximity to or touched against, e.g., receiver 106, prior to the start of a surgical procedure, such that user-identifying signal 250 from user identifier 130 may be transmitted to controller 104. In certain other embodiments, user identifier is a WiFi-enabled device that is able to transmit WiFi signals to controller 104 without user identifier 130 having to be present in operating environment 100.

In certain embodiments, user-identifying data is obtained by controller 104 via image-recognition of surgeon 110 using, e.g., imaging device 160, which may be communicatively coupled to surgical console 120 and/or controller 104. In such examples, imaging device 160 may capture and relay images of surgeon 110 for transmission to controller 104, which may utilize one or more image recognition algorithms to map the captured image(s) of surgeon 110 to a corresponding surgeon profile indicative of the identity of surgeon 110.

At operation 204, upon receipt of user-identifying data (e.g., signal 250), controller 104 maps the user-identifying data to a user profile of surgeon 110 and one or more corresponding and defined sets of parameters/settings for surgeon 110. The defined sets of parameters/settings may be surgeon-specific and may be used by surgical console 120 to operate and drive a corresponding surgical tool. For example, defined sets of parameters/settings may include user-defined (defined by surgeon 110) and user-preferred (preferred by surgeon 111) surgical tool and/or console settings, modes, sub-modes, task parameters, calibration data, and the like, for one or more different surgical tools and consoles, which may be predefined prior to performance of a surgical procedure. In certain embodiments, the defined sets of parameters/settings are stored within the user profile of the surgeon 110. In such embodiments, by mapping the user-identifying data to the surgeon profile, controller 104 is able to access surgeon 110's defined sets of parameters/settings. In certain embodiments, the defined sets of parameters/settings are stored within a memory of a user identifier, such as user identifier 130, and are transmitted to controller 104 with user-identifying signal 250.

In certain embodiments, at operation 206, upon mapping user-identifying signal 250 to a profile of surgeon 110 and one or more defined sets of sets of parameters/settings for surgeon 110, controller 104 may optionally request confirmation 252 from surgeon 110 to confirm a correct identification of surgeon 110 and/or the defined sets of parameters/settings. Requesting confirmation 252 may avoid incorrect identification of surgeon 110 and mapping to a non-corresponding user profile as well as parameters/settings, which may occur if more than one surgeon, or other operating staff, with their own user identifiers 130 are present in (or pass by) operating environment 100. Confirmation 252 may be in the form of an audible confirmation by surgeon 110, tactile confirmation, e.g., via pressing of a physical button or on-screen button, and the like. In certain embodiments, as part of confirmation 252, the mapped profile of surgeon 110 and/or the sets of parameters/settings are displayed on a display device for viewing by surgeon 110, such as display device 122 of surgical console 120. In certain embodiments, a pop-up window with one or more on-screen buttons may be displayed on display device 122 for surgeon 110 to confirm or disconfirm identification of surgeon 110 and/or the defined sets of parameters/settings by pressing of the on-screen buttons.

In certain embodiments, at operation 208, controller 104 optionally determines the operation mode of the surgical console 120. For example, surgical console 120 may have a number of different operation modes for different procedures, e.g., a “phaco” mode for performing phacoemulsification and related cataract surgery procedures, a vitreoretinal mode for performing vitreoretinal procedures (e.g., vitrectomy), etc. In such an example, controller 104 may determine the operation mode by mapping the user profile of surgeon 110 to a defined operation mode of surgical console 120 corresponding with surgeon 110. For example, for a surgeon 110 that performs phacoemulsification surgical procedures, controller 104 may determine that surgical console 120 needs to be placed in a phacoemulsification operation mode based on the profile of surgeon 110. Upon mapping to the operation mode of surgical console 120, controller 104 may cause surgical console 120 to be placed or switched into the mapped operation mode. In certain embodiments, however, controller 104 may request confirmation 256 of the defined operation mode by surgeon 110 prior to causing surgical console 120 to be placed or switched into the mapped operation mode, described below. Causing a surgical console 120 to be placed or switched into the mapped operation mode may comprise displaying a user interface associated with the mapped operation mode on display 122 of surgical console 120, unlocking or activating corresponding surgical tools 126 typically used for the mapped operation mode, etc.

In certain embodiments, at operation 210, upon determining an operation mode and prior to causing surgical console 120 to be placed or switched into the operation mode, controller 104 may optionally request confirmation 256 from surgeon 110 to confirm correct mapping and selection of the operation mode. Similar to confirmation 252, confirmation 254 may be in the form of an audible confirmation by surgeon 110, tactile confirmation, e.g., via pressing of a physical button or touch-screen button, and the like. In certain embodiments, as part of confirmation 255, the mapped operation mode is displayed on a display device for viewing by surgeon 110, such as display device 122 of surgical console 120. In certain embodiments, a pop-up window with one or more on-screen buttons may be displayed on display device 122 for surgeon 110 to confirm or disconfirm correct mapping and selection of the operation mode.

At operation 212, controller 104 receives tool-identifying data associated with surgical tool 126 that surgical console 120 is configured to drive. In certain embodiments, tool-identifying data is transmitted (e.g., wired or wirelessly) to controller 104 and/or to user identifier 130 in the form of a tool-identifying signal 258, which may indicate to controller 104 the type of tool, device, model, etc. of surgical tool 126. In certain embodiments, tool-identifying signal 258 is transmitted to controller from tool identifier 140 associated with surgical tool 126. As shown in FIG. 2B, tool identifier 140 may directly or indirectly interface with controller 104 and in certain embodiments, user identifier 130. As described above, tool identifier 140 generally includes any suitable component or device that may transmit tool-identifying signal 258 to controller 104 and/or user identifier 130 relating to the identity of surgical tool 126 for purposes of setup and configuration of surgical console 120, according to defined parameters/settings associated with surgical tool 126. In certain embodiments, surgical tool 126 may include a usage sensor (either as part of tool identifier 140 or separately) for generating sensory signals as a result of surgical tool 126 being used. For example, the usage sensor may include any suitable type of sensor, component, or device for detecting usage or handling 260 of surgical tool 126 by surgeon 110. In certain embodiments, tool identifier 140 transmits tool-identifying signal 258 to controller 104 and/or user identifier 130 upon the usage sensor generating sensory signals indicating that surgical tool 126 is being used.

In certain embodiments, tool-identifying signal 258 is transmitted to controller 104 directly from surgical tool 126, e.g., upon detection of usage or handling 260 of surgical tool 126 by the usage sensor. In certain other embodiments, however, tool-identifying signal 258 is first transmitted by surgical tool 126 to user identifier 130, which then relays tool-identifying signal 258 to controller 104. For example, user identifier 130 may be an RFID-type bracelet, and tool identifier 140 may be a similar RFID-type component on surgical tool 126. Close proximity of the RFID-type bracelet to tool identifier 140 (as a result of surgeon 110 grabbing surgical tool 126) may signal usage or handling 260 of the tool and cause tool-identifying signal 258 to be transmitted from tool identifier 140 to user identifier 130. User identifier 130 may, in turn, relay tool-identifying signal 258 to controller 104 (e.g., along with user-identifying signal 250) thereby indicating to controller 104 that surgeon 110 is using surgical tool 126.

In certain embodiments, instead of or in addition to surgical tool 126 having a usage sensor, a usage sensor (e.g., laser sensor, load sensor, etc.) may be positioned on or be provided as part of surgical console 120. In such embodiments, the usage sensor may be configured to generate sensory signals when a user picks up surgical tool 126 from its at-rest position on surgical console 120. In certain embodiments, the sensory signals may then cause controller 104 to receive tool-identifying signal 258 for identifying surgical tool 126. In embodiments where a usage sensor is positioned on or provided by surgical console 120, the sensory signals generated as a result of handling surgical tool 126 may act as tool-identifying signal 258.

In certain embodiments, tool-identifying data is obtained via image-recognition of surgical tool 126 using, e.g., imaging device 160, which may be communicatively coupled to surgical console 120 and/or controller 104. In such examples, imaging device 160 may capture and relay images of surgical tool 126 for transmission to controller 104, which may then utilize one or more image recognition algorithms to map the captured image(s) of surgical tool 126 to corresponding tool-identifying data.

At operation 214, upon receipt of tool-identifying data (e.g., signal 258), controller 104 maps the tool-identifying data to a tool profile. For example, if surgical tool 126 is a vitreoretinal probe, then the tool-identifying data maps to a vitreoretinal probe profile. In certain embodiments, once a tool profile is mapped to, controller 104 maps the identification of surgical tool 126 (as indicated by the tool profile) to one of the one or more surgeon-specific sets of parameters/settings previously mapped at operation 204, and/or an operation mode of surgical console 120. For example, the surgeon-specific parameters/settings mapped to at operation 204 may include parameters/settings for performing a vitreoretinal procedure, as well as parameters/settings for performing a phaco procedure. In such an example, identification of surgical tool 126 may be used by controller 104 to determine which of the one or more sets of parameters/settings are applicable. For example, if the identified surgical tool 126 is a vitreoretinal probe, then controller 104 identifies that the parameters/settings for performing a vitreoretinal procedure are to be used when surgical tool 126 is being used by surgeon 110.

In addition, in cases where controller 104 is not able to select an operation mode based on the surgeon profile (e.g., if surgeon performs various procedures corresponding with different operation modes), then the tool-identifying data may be used by controller 104 to map to an appropriate operation mode. For example, if surgical tool 126 that surgeon 110 has just picked up is a vitreoretinal probe, then controller 104 may determine that surgical console 120 should be placed or switched into an operation mode associated with vitreoretinal surgery.

In certain embodiments, at operation 216, controller 104 may optionally request confirmation 262 from surgeon 110 to confirm correct identification of surgical tool 126. Requesting confirmation 262 may avoid incorrect identification of surgical tool 126 and mapping to a non-corresponding tool profile, which may occur if more than one surgical tool is present in operating environment 100. Confirmation 262 may be in the form of an audible confirmation by surgeon 110, tactile confirmation, e.g., via pressing of a physical button or touch-screen button, and the like. In certain embodiments, as part of confirmation 262, an image of the identified surgical tool 126, or the mapped tool profile, are displayed on a display device for viewing by surgeon 110, such as display device 122 of surgical console 120. In certain embodiments, a pop-up window with one or more on-screen buttons may be displayed on display device 122 for surgeon 110 to confirm or disconfirm correct identification of surgical tool 126.

At operation 218, controller 104 drives surgical tool 126 according to the surgeon-specific parameters/settings in the mapped operation mode. For example, controller 104 first places surgical console 120 in the mapped operation mode (e.g., vitreoretinal mode), which, as an example, may cause surgical console 120 to display a user interface (UI) associated with the operation mode (e.g., vitreoretinal-related UI) on display 122. Then, in response to a trigger (surgeon-initiated trigger e.g., through a foot pedal, etc.) controller 104 may initiate driving of surgical tool 126 according to the surgeon-specific parameters/settings. As described above, the surgeon-specific parameters/settings may include surgical tool and/or console settings, modes, sub-modes, task parameters, calibration data, and the like. For example, in examples where surgical tool 126 is a vitreoretinal probe, controller 104 may initiate driving surgical tool 126 according to surgeon-specific vitreoretinal tool sub-modes, each of which may include different duty cycles, minimum and maximum cut-rate and/or vacuum thresholds, and the like. In certain embodiments, driving of surgical tool 126 may be triggered or associated with surgeon-initiated travel of a foot pedal or similar device in communication with surgical console 120.

FIG. 3 illustrates an exemplary diagram showing how various components of operating environment 100, shown in FIGS. 1-2, communicate and operate together. As shown, surgical console 120 includes, without limitation, controller 104 and receiver 106, which enable connection of controller 104 to user identifier 130 and/or tool identifier 140. Controller 104 includes interconnect 310 and network interface 312 for connection with data communications network 350. Controller 104 further includes central processing unit (CPU) 316, memory 318, and storage 320. CPU 316 may retrieve and store application data in the memory 318, as well as retrieve and execute instructions stored in the memory 318. Interconnect 310 transmits programming instructions and application data among CPU 316, network interface 312, memory 318, storage 320, surgical tool 126, and imaging device 360, etc. CPU 316 can represent a single CPU, multiple CPUs, a single CPU having multiple processing cores, and the like. Memory 318 represents random access memory.

Storage 320 may be a disk drive. Although shown as a single unit, storage 320 may be a combination of fixed or removable storage devices, such as fixed disc drives, removable memory cards or optical storage, network attached storage (NAS), or a storage area-network (SAN). Storage 320 may comprise profiles 332 of users, e.g., surgeon 110, within an operating environment that may utilize, e.g., surgical console 120, and each user profile 332 may include user-specific parameters/settings 334 and/or mappings of the user identity to one or more operation modes. Storage 320 may further include tool profiles 338, each indicating the corresponding type of tool and/or mappings to one or more operation modes. Storage 320 may further include operation modes 336, each mode having pre-set instructions for operating surgical console 120 in the corresponding operation mode.

Memory 318 comprises configuration module 322 that includes instructions, which when executed by CPU 316, allow controller 104 to identify a user and/or a tool in the operating environment, as described in the embodiments herein. Memory 318 may also include an operating system and/or one or more applications (not shown), which when executed by CPU 316, allow controller 104 to operate surgical console 120 (e.g., including driving tool 126 based on retrieved parameters/settings). For example, according to embodiments described herein, memory 318 includes user identification module 324 which comprises executable instructions for identifying a user via user identifier 130, and for mapping the user to user profile 332. In addition, memory 318 includes tool identification module 326, which comprises executable instructions for identifying the type of surgical tool 126, and mapping it to a corresponding tool profile 338.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein, but are to be accorded the full scope consistent with the language of the claims.

Within a claim, reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for.” The word “exemplary” is used herein to mean “serving as an example, instance, or illustration.” Any aspect described herein as “exemplary” is not necessarily to be construed as preferred or advantageous over other aspects.

What is claimed is:

1. A system for configuring a surgical console in a surgical operating environment, comprising:
a memory comprising executable instructions; and
a processor in data communication with the memory and configured to execute the instructions to cause the system to:
receive user-identifying information associated with a user in the surgical operating environment;
map the user-identifying information to a user profile;
identify, based on the user profile, one or more parameters for driving a surgical tool; and
configure the surgical console to drive the surgical tool based on the one or more parameters.

2. The system of claim 1, further configured to display the one or more parameters on a display device of the surgical console.

3. The system of claim 1, wherein the user-identifying information is received from an identification tag or identification badge of the user.

4. The system of claim 3, wherein the identification tag or identification badge comprises a radio frequency identification (RFID) device configured to send a signal comprising the user-identifying information, and wherein the processor is in data communication with an RFID reader configured to receive the signal and relay the user-identifying information to the processor.

5. The system of claim 1, wherein the user-identifying information is received as an encrypted communication signal from a smart device of the user.

6. The system of claim 1, wherein the user-identifying information comprises an image of the user captured by an imaging device in data communication with the processor, and wherein the processor utilizes one or more image recognition algorithms to map the captured image of the user to the user profile.

7. The system of claim 1, wherein executing the instructions further causes the system to:
receive tool-identifying information associated with the surgical tool; and
map the tool-identifying information to a tool profile, wherein the identification of the one or more parameters is further based on the tool profile.

8. The system of claim 7, further configured to display the tool profile on a display device of the surgical console.

9. The system of claim 7, wherein the tool-identifying information is received from a tool identifier on the surgical tool.

10. The system of claim 9, wherein the tool identifier comprises a radio frequency identification (RFID) device.

11. The system of claim 9, wherein the surgical tool comprises a sensor, the sensor configured to initiate sending of the tool-identifying information from the tool identifier upon the surgical tool being held by the user or the sensor being pressed.

12. The system of claim 7, wherein the tool-identifying information comprises an image of the surgical tool captured by an imaging device in data communication with the processor, and wherein the processor utilizes one or more image recognition algorithms to map the captured image to the tool profile.

13. The system of claim 12, wherein the user-identifying information comprises an image of the user captured by the imaging device, wherein the processor utilizes the one or more image recognition algorithms to map the captured image of the user to the user profile, and wherein the processor further utilizes the one or more image recognition algorithms to determine whether the surgical tool is being used by the user.

14. The system of claim 1, wherein executing the instructions further causes the system to:
identify, based on the user profile, an operation mode of the surgical console associated with the user; and
place the surgical console in the operation mode.

15. The system of claim 14, further configured to display the operation mode associated with the user on a display device of the surgical console.
